(19) United States
(12) Patent Application Publication
(10) Pub. No.: US 2014/0107080 A1
(43) Pub. Date: Apr. 17, 2014

(54) BRAIN FUNCTION IMPROVING AGENT

(71) Applicant: NIPPON SUISAN KAISHA, LTD, Chiyoda, Tokyo (JP)

(72) Inventors: Yoshihiko Koga, Mitaka-shi (JP); Chizuru Konagai, Mitaka-shi (JP); Kenichi Yanagimoto, Chiyoda-ku (JP); Kohsuke Hayamizu, Chiyoda-ku (JP)

(73) Assignee: NIPPON SUISAN KAISHA, LTD., Chiyoda-ku, Tokyo (JP)

(21) Appl. No.: 14/122,466

(22) PCT Filed: May 31, 2012
(86) PCT No.: PCT/JP2012/064186

§ 371 Date: Nov. 26, 2013

(30) Foreign Application Priority Data
May 31, 2011 (JP) 121269/2011
May 31, 2011 (JP) 121405/2011

(51) Int. Cl. A61K 35/56; A23L 1/30; A61K 31/683

(52) U.S. Cl. CPC A61K 35/612; A61K 31/683; A23L 1/3006
USPC 514/121

(57) ABSTRACT

Brain function improving agents, cognitive judgment ability improving agents, as well as foods and beverages that contain krill oil as an active ingredient are provided.

### TECHNICAL FIELD

The present invention relates to brain function improving agents as well as foods and beverages for improving brain functions. The present invention also relates to cognitive judgment ability improving agents as well as foods and beverages for improving cognitive judgment ability.

### BACKGROUND ART

Recent years are seeing a growing interest in the effects that food ingredients might have on brain functions. For example, non-invasive methods such as near-infrared spectroscopy (NIRS) are used to study the effects that food ingredients might have on brain functions during the execution of intellectual tasks.

A report published concerning the effect of protecting brain functions states that astaxanthin and/or an ester thereof prevents cerebral infarction by suppressing the generation of active oxygen within the brain (Patent Document 1.) Another report published concerning an improvement of brain functions states that flavor components in whiskey or brandy suppress the elevation of blood flow within the brain to increase the efficiency of brain's processing function during the execution of a verbal fluency task (Patent Document 2.)

In addition, electroencephalogram analyses as of background electroencephalographic activity and event-related potentials (ERPs) are made to study the effects that food-derived components might have on brain functions, in particular, the effects that they might have on brain functions during the execution of intellectual tasks.

For example, a report has been published to show that triglycerides having docosahexaenoic acid (DHA) as a constituent fatty acid accelerate the peak latency of P300 as a component of event-related potentials in healthy persons and patients with dementia to improve cognitive response functions (Patent Document 3.) It has also been reported that arachidonic acid or compounds having arachidonic acid as a constituent fatty acid prevent, ameliorate or improve a decline in the normal response of cognitive judgment ability (Patent Document 4.) Moreover, it has been reported that astaxanthin or esters thereof improve cognitive behavioral ability (Patent Document 5.)

### CITATION LIST

### Patent Documents

Patent Document 1: Japanese Unexamined Patent Application Publication No. 2007-314436

Patent Document 2: Japanese Unexamined Patent Application Publication No. 2006-022036

Patent Document 3: Japanese Unexamined Patent Application Publication No. Hei 10-59844

Patent Document 4: Published Japanese Translation of PCT International Publication for Patent Application No. 2006-502196

Patent Document 5: Japanese Unexamined Patent Application Publication No. 2010-270095

### SUMMARY OF INVENTION

### Technical Problem

However, no food-derived components are known that can improve brain functions by promoting the cerebral metabolism during the execution of tasks. An object of the present invention is to provide brain function improving agents and foods for improving brain functions.

Fatty acids are present in the living body and foods primarily as constituent fatty acids of triglycerides or phospholipids. However, it is not known that fatty acids produce different effects on cognitive judgment ability depending on the form in which they are present. Another object of the present invention is to provide cognitive judgment ability improving agents as well as foods and beverages that may be ingested for improving cognitive judgment ability.

### Solution to Problem

In view of the above-described circumstances, the present inventors paid particular attention to krill oil which is abundant in phospholipids. As a result of their intensive studies, the present inventors found that subjects continuously allowed to ingest the krill oil had their brain functions improved. The present invention has been accomplished on the basis of this finding.

The present invention provides brain function improving agents as recited below under (1) to (4).

(1) A brain function improving agent which contains krill oil as an active ingredient.
(2) The brain function improving agent as recited in (1), wherein the krill oil is purified via a thermal coagulum of krill.
(3) The brain function improving agent as recited in (1) or (2), wherein an n-3 polyunsaturated fatty acid accounts for at least 5% (w/w) of the fatty acid composition of the krill oil.
(4) The brain function improving agent as recited in any one of (1) to (3), which is for administering the krill oil to a subject in a dose of 0.02-400 mg/kg body weight/day.

According to another aspect, the present invention provides a food or beverage as recited below under (5).

(5) A food or beverage containing the brain function improving agent as recited in any one of (1) to (4).

According to yet another aspect, the present invention provides brain function improving agents as recited under (6) to (12).

(6) A brain function improving agent which contains a phospholipid having a polyunsaturated fatty acid as a constituent fatty acid.
(7) The brain function improving agent as recited in (6), wherein the polyunsaturated fatty acid is an n-3 polyunsaturated fatty acid.
(8) The brain function improving agent as recited in (7), wherein the n-3 polyunsaturated fatty acid is eicosapentaenoic acid or docosahexenoic acid.
(9) The brain function improving agent as recited in (7) or (8), wherein the n-3 polyunsaturated fatty acid accounts for at least 5% (w/w) of the constituent fatty acids in the phospholipid.
(10) The brain function improving agent as recited in (8) or (9), wherein eicosapentaenoic acid accounts for at least 4% (w/w) of the constituent fatty acids in the phospholipid.
(11) The brain function improving agent as recited in any one of (8) to (10), wherein docosahexaenoic acid accounts for at least 3% (w/w) of the constituent fatty acids in the phospholipid.
(12) The brain function improving agent as recited in any one of (6) to (11), wherein the phospholipid is selected from the group consisting of phosphatidylcholine, phosphatidylserine, phosphatidylethanolamine, phosphatidic acid, phosphatidylglycerol, and phosphatidylinositol.

According to another aspect, the present invention provides a food or beverage as recited below under (13).

(13) A food or beverage containing the brain function improving agent as recited in any one of (6) to (12).

According to another aspect, the present invention provides brain function improving agents or foods or beverages as recited below under (14) to (20).

(14) The brain function improving agent or the food or beverage as recited in any one of (1) to (13), which is for ingestion before or during the execution of a task selected from the group consisting of memorization, calculation, reading a book, writing a composition, dissertation, and argumentation.
(15) The brain function improving agent or the food or beverage as recited in any one of (1) to (13), which is for increasing the percentage of examination questions answered correctly.
(16) The brain function improving agent or the food or beverage as recited in any one of (1) to (13), which is for ingestion by a subject in need of an improvement of brain functions.
(17) The brain function improving agent or the food or beverage as recited in any one of (1) to (13), which is for ingestion by a person engaged in an intellectual task.
(18) The brain function improving agent or the food or beverage as recited in any one of (1) to (13), which is for ingestion during working hours.
(19) The food or beverage as recited in (5) or (13), which is to be used for school lunch.
(20) A food or beverage as recited in (5) or (13), which is for lunch.

According to another aspect, the present invention provides foods or beverages as recited below under (21) to (32).

(21) A food or beverage containing krill oil, which is for ingestion before or during the execution of a task selected from the group consisting of memorization, calculation, reading a book, writing a composition, dissertation, and argumentation.
(22) A food or beverage containing krill oil, which is for increasing the percentage of examination questions answered correctly.
(23) A food or beverage containing krill oil, which is for ingestion by a subject in need of an improvement of brain functions.
(24) A food or beverage containing krill oil, which is for ingestion by a person engaged in an intellectual task.
(25) A food or beverage containing krill oil, which is for ingestion during working hours.
(26) A food or beverage containing krill oil, which is to be used for school lunch
(27) A food or beverage containing krill oil, which is for lunch.
(28) The food or beverage as recited in any one of (21) to (27), wherein the krill oil is purified via a thermal coagulum of krill.
(29) The food or beverage as recited in any one of (21) to (28), wherein an n-3 polyunsaturated fatty acid accounts for at least 5% (w/w) of the fatty acid composition of the krill oil.
(30) The food or beverage as recited in any one of (21) to (29), wherein eicosapentaenoic acid accounts for at least 2% (w/w) of the fatty acid composition of the krill oil.
(31) The food or beverage as recited in any one of (21) to (30), wherein docosahexaenoic acid accounts for at least 1% (w/w) of the fatty acid composition of the krill oil.
(32) The food or beverage as recited in any one of (21) to (31), which is for administering the krill oil to a subject in a dose of 0.02-400 mg/kg body weight/day.

According to another aspect, the present invention provides foods or beverages as recited (33) to (45).

(33) A food or beverage containing a phospholipid, which is for ingestion before or during the execution of a task selected from the group consisting of memorization, calculation, reading a book, writing a composition, dissertation, and argumentation.
(34) A food or beverage containing a phospholipid, which is for increasing the percentage of examination questions answered correctly.
(35) A food or beverage containing a phospholipid, which is for ingestion by a subject in need of an improvement of brain functions.
(36) A food or beverage containing a phospholipid, which is for ingestion by a person engaged in an intellectual task.
(37) A food or beverage containing a phospholipid, which is for ingestion during working hours.
(38) A food or beverage containing a phospholipid, which is to be used for school lunch.
(39) A food or beverage containing a phospholipid, which is for lunch.
(40) The food or beverage as recited in any one of (33) to (39), wherein the phospholipid has a polyunsaturated fatty acid as a constituent fatty acid.
(41) The food or beverage as recited in (40), wherein the polyunsaturated fatty acid is an n-3 polyunsaturated fatty acid.
(42) The food or beverage as recited in (41), wherein the n-3 polyunsaturated fatty acid is eicosapentaenoic acid or docosahexenoic acid.
(43) The food or beverage as recited in (42), wherein eicosapentaenoic acid accounts for at least 4% (w/w) of the constituent fatty acids in the phospholipid.
(44) The food or beverage as recited in (42) or (43), wherein docosahexaenoic acid accounts for at least 3% (w/w) of the fatty acid composition of the phospholipid.
(45) The food or beverage as recited in any one of (33) to (44), wherein the phospholipid is selected from the group consisting of phosphatidylcholine, phosphatidylserine, phosphatidylethanolamine, phosphatidic acid, phosphatidylglycerol, and phosphatidylinositol.

According to another aspect, the present invention provides a method as recited below under (46).

(46) A method of improving brain functions which comprises ingesting the brain function improving agent or the food or beverage as recited in any one of (1) to (45).

According to another aspect, the present invention provides use as recited below under (47).

(47) Use of krill oil or phospholipid in the manufacture of a pharmaceutical drug for improving brain functions.

The present inventors further paid particular attention to krill oil which is abundant in phospholipids. As a result of their intensive studies, the present inventors found that subjects continuously allowed to ingest the krill oil had their cognitive judgment ability improved. The present invention has been accomplished on the basis of this finding.

The present invention provides cognitive judgment ability improving agents as recited below under (48) to (51).

(48) A cognitive judgment ability improving agent which contains krill oil as an active ingredient.
(49) The cognitive judgment ability improving agent as recited in (48), wherein the krill oil is purified via a thermal coagulum of krill.
(50) The cognitive judgment ability improving agent as recited in (48) or (49), wherein an n-3 polyunsaturated fatty acid accounts for at least 5% (w/w) of the fatty acid composition of the krill oil.
(51) The cognitive judgment ability improving agent as recited in any one of (48) to (50), which is for administering the krill oil to a subject in a dose of 0.02-400 mg/kg body weight/day.

According to another aspect, the present invention provides a food or beverage as recited under (52).

(52) A food or beverage containing the cognitive judgment ability improving agent as recited in any one of (48) to (51).

According to another aspect, the present invention provides cognitive judgment ability improving agents as recited under (53) to (59).

(53) A cognitive judgment ability improving agent which contains a phospholipid having a polyunsaturated fatty acid as a constituent fatty acid.
(54) The cognitive judgment ability improving agent as recited in (53), wherein the polyunsaturated fatty acid is an n-3 polyunsaturated fatty acid.
(55) The cognitive judgment ability improving agent as recited in (54), wherein the n-3 polyunsaturated fatty acid is eicosapentaenoic acid or docosahexenoic acid.
(56) The cognitive judgment ability improving agent as recited in (54) or (55), wherein the n-3 polyunsaturated fatty acid accounts for at least 5% (w/w) of the constituent fatty acids in the phospholipid.
(57) The cognitive judgment ability improving agent as recited in (55) or (56), wherein eicosapentaenoic acid accounts for at least 4% (w/w) of the constituent fatty acids in the phospholipid.
(58) The cognitive judgment ability improving agent as recited in any one of (55) to (57), wherein docosahexaenoic acid accounts for at least 3% (w/w) of the constituent fatty acids in the phospholipid.
(59) The cognitive judgment ability improving agent as recited in any one of (53) to (58), wherein the phospholipid is selected from the group consisting of phosphatidylcholine, phosphatidylserine, phosphatidylethanolamine, phosphatidic acid, phosphatidylglycerol, and phosphatidylinositol.

According to another aspect, the present invention provides a food or beverage as recited under (60).

(60) A food or beverage containing the cognitive judgment ability improving agent as recited in any one of (53) to (59).

According to another aspect, the present invention provides cognitive judgment ability improving agents or foods or beverages as recited under (61) to (67).

(61) The cognitive judgment ability improving agent or the food or beverage as recited in any one of (48) to (60), which is for ingestion before or during a work that requires quick judgment on a new task.
(62) The cognitive judgment ability improving agent or the food or beverage as recited in any one of (48) to (60), which is for ingestion before or during exercise.
(63) The cognitive judgment ability improving agent or the food or beverage as recited in any one of (48) to (60), which is for ingestion before or during the driving of a vehicle that requires operation control as exemplified by a bicycle, motorcycle, airplane, train, or an automobile.
(64) The cognitive judgment ability improving agent or the food or beverage as recited in any one of (48) to (60), which is for ingestion by a subject in need of an improvement in cognitive judgment ability.
(65) The cognitive judgment ability improving agent or the food or beverage as recited in any one of (48) to (60), which is for ingestion during working hours.
(66) The food or beverage as recited in (52) or (56), which is for school lunch.
(67) The food or beverage as recited in (52) or (60), which is for lunch.

According to another aspect, the present invention provides foods or beverages as recited under (68) to (79).

(68) A food or beverage containing krill oil, which is for ingestion before or during a work that requires quick judgment on a new task.
(69) A food or beverage containing krill oil, which is for ingestion before or during exercise.
(70) A food or beverage containing krill oil, which is for ingestion before or during the driving of a vehicle that requires operation control as exemplified by a bicycle, motorcycle, airplane, train, or an automobile.
(71) A food or beverage containing krill oil, which is for ingestion by a subject in need of an improvement in cognitive judgment ability.
(72) A food or beverage containing krill oil, which is for ingestion during working hours.
(73) A food or beverage containing krill oil, which is for school lunch.
(74) A food or beverage containing krill oil, which is for lunch.
(75) The food or beverage as recited in any one of (68) to (74), wherein the krill oil is purified via a thermal coagulum of krill.
(76) The food or beverage as recited in any one of (68) to (75), wherein an n-3 polyunsaturated fatty acid accounts for at least 5% (w/w) of the fatty acid composition of the krill oil.
(77) The food or beverage as recited in any one of (68) to (76), wherein eicosapentaenoic acid accounts for at least 2% (w/w) of the fatty acid composition of the krill oil.
(78) The food or beverage as recited in any one of (68) to (77), wherein docosahexaenoic acid accounts for at least 1% (w/w) of the fatty acid composition of the krill oil.
(79) The food or beverage as recited in any one of (68) to (78), which is for administering the krill oil to a subject in a dose of 0.02-400 mg/kg body weight/day.

According to another aspect, the present invention provides foods or beverages as recited under (80) to (93).

(80) A food or beverage containing a phospholipid, which is for ingestion before or during a work that requires quick judgment on a new task.
(81) A food or beverage containing a phospholipid, which is for ingestion before or during exercise.
(82) A food or beverage containing a phospholipid, which is for ingestion before or during the driving of a vehicle that requires operation control as exemplified by a bicycle, motorcycle, airplane, train, or an automobile.
(83) A food or beverage containing a phospholipid, which is to be ingested by a subject in need of an improvement in cognitive judgment ability.
(84) A food or beverage containing a phospholipid, which is for ingestion during working hours.
(85) A food or beverage containing a phospholipid, which is for school lunch
(86) A food or beverage containing a phospholipid, which is for lunch.
(87) The food or beverage as recited in any one of (80) to (86), wherein the phospholipid has a polyunsaturated fatty acid as a constituent fatty acid.
(88) The food or beverage as recited in (87), wherein the polyunsaturated fatty acid is an n-3 polyunsaturated fatty acid.
(89) The food or beverage as recited in (88), wherein the n-3 polyunsaturated fatty acid is eicosapentaenoic acid or docosahexenoic acid.
(90) The food or beverage as recited in (88) or (89), wherein the n-3 polyunsaturated fatty acid accounts for at least 5% (w/w) of the constituent fatty acids in the phospholipid.
(91) The food or beverage as recited in (89) or (90), wherein eicosapentaenoic acid accounts for at least 4% (w/w) of the constituent fatty acids in the phospholipid.
(92) The food or beverage as recited in any one of (89) to (91), wherein docosahexaenoic acid accounts for at least 3% (w/w) of the constituent fatty acids in the phospholipid.
(93) The food or beverage as recited in any one of (80) to (92), wherein the phospholipid is selected from the group consisting of phosphatidylcholine, phosphatidylserine, phosphatidylethanolamine, phosphatidic acid, phosphatidylglycerol, and phosphatidylinositol.

According to another aspect, the present invention provides a method as recited under (94).

(94) A method of improving cognitive judgment ability which comprises ingesting the cognitive judgment ability improving agent or the food or beverage as recited in any one of (48) to (93).

According to another aspect, the present invention provides use as recited under (95).

(95) Use of krill oil or phospholipid in the manufacture of a pharmaceutical drug for improving cognitive judgment ability.

### Advantageous Effects of Invention

According to the present invention, the level of oxyhemoglobin within the brain during the execution of a task is increased to promote the cerebral metabolism, whereby brain functions can be improved. This effect works advantageously in performing tasks that require logical thinking, as exemplified by memorization, calculation, reading a book, writing a composition, dissertation, and argumentation. It also works advantageously in recovering brain functions that have declined due to fatigue, aging or disease, as well as in preventing such decline.

As a further advantage of the present invention, the latency of P300 that appears as one component of event-related potentials in response to a newly presented task can be shortened. For example, by ingesting the cognitive judgment ability improving agent or a food or beverage of the present invention, the latency of P300 can be made shorter by at least 3 milliseconds than before it was ingested. The difference of 20 milliseconds in terms of the latency of P300 is said to correspond to a human age difference of 20 years in terms of the speed at which a task is processed.

Thus, by ingesting the cognitive judgment ability improving agent of the present invention or a food or beverage that contains it, the speed at which a newly presented task is processed can be increased and, hence, the present invention contributes to improvements as in the athlete's competitive ability in sports and the ability to avoid a danger during the operation control of a vehicle. It also works advantageously in recovering cognitive judgment ability that has declined due to fatigue, aging or disease, as well as preventing such decline.

### BRIEF DESCRIPTION OF DRAWINGS

FIG. 1 is a diagram showing sites of measurement by NIRS; an irradiation probe or a detection probe was attached to each of the locations indicated by numbers 1 to 24.

FIG. 2 is a set of charts showing changes in the level of oxyhemoglobin within the brain during execution of a 2-back test. Results on a subject at the 12^{th} week of sample ingestion are shown; ch (channel) 1 to 24 in the figure represent the sites of measurement on the head and correspond to the positions of numbers 1 to 24 in FIG. 1; the horizontal axis of each chart represents the time of measurement and the vertical axis represents the level of oxyhemoglobin; the 2-back test was conducted for 450 seconds.

FIG. 3 shows the level of oxyhemoglobin within the brain at the point in time of 225.0 seconds after the start of the 2-back test on a subject at the 12^{th} week of sample ingestion; the level of oxyhemoglobin is shown by color and the more red the color is, the higher the oxyhemoglobin level whereas the more blue the color is, the lower the oxyhemoglobin level.

FIG. 4 is set of charts showing changes in the level of oxyhemoglobin within the brain during execution of a Kraepelin test. Results on a subject at the 12^{th} week of sample ingestion are shown; ch (channel) 1 to 24 in the figure represent the sites of measurement on the head and correspond to the positions of numbers 1 to 24 in FIG. 1; the horizontal axis of each chart represents the time of measurement and the vertical axis represents the level of oxyhemoglobin; the Kraepelin test was conducted for 300 seconds.

FIG. 5 shows the level of oxyhemoglobin within the brain at the point in time of 150.0 seconds after the start of the Kraepelin test on a subject at the 12^{th} week of sample ingestion; the level of oxyhemoglobin is shown by color and the more red the color is, the higher the oxyhemoglobin level whereas the more blue the color is, the lower the oxyhemoglobin level.

FIG. 6 is a graph showing the number of incorrect answers in the Kraepelin test conducted at the 12^{th} week of sample ingestion; MC, medium chain fatty acid oil ingestion group; SO, sardine oil ingestion group; KO, krill oil ingestion group.

FIG. 7 shows changes in the peak latency of P300 in Cz region (A) and Pz region (B) among the medium chain fatty acid oil ingestion group, the sardine oil ingestion group, and the krill oil ingestion group. Changes in the peak latency of P300 at the 6^{th} and 12^{th} weeks after sample ingestion are shown relative to the value before sample ingestion; , medium chain fatty acid oil ingestion group; Δ, sardine oil ingestion group; ◯, krill oil ingestion group.

### DESCRIPTION OF EMBODIMENTS

On the following pages, the present invention is described more specifically.

The present invention provides a brain function improving agent that contains krill oil. The brain function improving agent of the present invention contains an effective amount of krill oil. The “effective amount” as referred to herein means the necessary amount for improving brain functions. For example, the daily amount of krill oil to be ingested is 0.02 to 400 mg/kg, preferably 10-200 mg/kg, more preferably 16-100 mg/kg, and particularly preferably 20-80 mg/kg, for example, 25-40 mg/kg, per kilogram of an animal's body weight. The “animal” as referred to herein includes, for example, mammals including human, bovine, equine, swine, sheep, goat, donkey, camel, llama, elephant, alpaca, reindeer (caribou), zebu, Bubalus arnee, yak, guinea pig, rabbit, mink, chicken, duck, goose, turkey, Cairina moschata, quail, ostrich, domestic pigeon, pheasant, Phalacrocorax capillatus, canine, feline, hamster, guinea pig, ferret, squirrel, monkey, etc. and fishes including sea bream, tuna, yellow tail, great amberjack, Seriola lalandi, horse mackerel, mackerel, bass, eel, flounder, sole, puffer, salmon, trout, catfish, grouper, barramundi, cobia, etc.

In particular, the amount to be ingested by a human adult is 1-20000 mg/50 kg body weight, preferably 500-10000 mg/50 kg body weight, more preferably 80-5000 mg/50 kg body weight, and particularly preferably 1000-4000 mg/50 kg body weight, for example, 1250-2000 mg/50 kg body weight, per day. In order to ensure that a more marked effect is obtained for improving brain functions in a human adult, the amount of ingestion is preferably increased but if it is excessive, increased oiliness results to cause undesirable effects including retarded absorption, indigestion, dyspepsia, and avoidance of ingestion. The amounts of ingestion indicated above may refer to a single ingestion or multiple, for example, two or three ingestions. It should also be noted that the brain function improving agent of the present invention is preferably ingested continuously; for example, it is continuously ingested for at least 7 days, preferably at least 30 days, and more preferably at least 90 days.

The present invention also provides a cognitive judgment ability improving agent that contains krill oil.

The cognitive judgment ability improving agent of the present invention contains an effective amount of krill oil. The “effective amount” as referred to herein means the necessary amount for improving cognitive judgment ability. For example, the daily amount of krill oil to be ingested is 0.02 to 400 mg/kg, preferably 10-200 mg/kg, more preferably 16-100 mg/kg, and particularly preferably 20-80 mg/kg, for example, 25-40 mg/kg, per kilogram of an animal's body weight.

In particular, the amount to be ingested by a human adult is 1-20000 mg/50 kg body weight, preferably 500-10000 mg/50 kg body weight, more preferably 800-5000 mg/50 kg body weight, and particularly preferably 1000-4000 mg/50 kg body weight, for example, 1250-2000 mg/50 kg body weight, per day. In order to ensure that a more marked effect is obtained for improving cognitive judgment ability in a human adult, the amount of ingestion is preferably increased but if it is excessive, increased oiliness results to cause undesirable effects including retarded absorption, indigestion, dyspepsia, and avoidance of ingestion. The amounts of ingestion indicated above may refer to a single ingestion or multiple, for example, two or three ingestions. It should also be noted that the cognitive judgment ability improving agent of the present invention is preferably ingested continuously; for example, it is continuously ingested for 7-30 days, preferably 30-90 days, and more preferably at least 90 days.

The “krill” as referred to herein may be an arthropod belonging to the phylum Arthropoda, subphylum Crutacea, class Malacostraca, and examples include arthropods belonging to the phylum Arthropoda, subphylum Crutacea, class Malacostraca, order Eucarida, family Euphausiacea, such as antarctic krill (Euphausia superba), and arthropods belonging to the phylum Arthropoda, subphylum Crutacea, class Malacostraca, order Peracarida, family Mysidae, such as mysids caught in the seas around Japan. It should, however, be noted that antarctic krill is particularly preferred from the viewpoints of stability in catch and uniformity in the quality of the lipid component. The krill oil as referred to herein means the one that is obtained from the above-mentioned types of krill.

The krill oil to be used in the present invention can be obtained by known production methods. For example, it can be produced by referring to the known methods described in WO 2000/023546 A1, WO 2009/027692 A1, WO 2010/035749 A1, WO 2010/035750 A1, etc. At least the krill oil that can be produced by the methods described in these international publications can preferably be used in the present invention.

The krill oil to be used in the present invention can be prepared by extraction with suitable organic solvents from a solids content as a krill-derived starting material in accordance with, for example, the methods described in the international publications identified above. Suitable organic solvents include, for example, alcohols such as methanol, ethanol, propanol, isopropanol, butanol, propylene glycol, and butylene glycol, as well as methyl acetate, ethyl acetate, acetone, chloroform, toluene, pentane, hexane, cyclohexane, etc., which may be used either alone or in combination of two or more species. When these solvents are used, their mixing ratio or the proportion between raw material and solvent may be set at any desired value.

The above-mentioned solids content as the krill-derived starting material may be in the form of dried krill, milled krill, raw krill or frozen krill; alternatively, either whole krill or a part thereof may be pressed into a liquid form and the resulting pressed liquid is heated to separate a solids content from water-soluble components. The pressing operation can be performed with commonly employed apparatuses, including a hydraulic press, a screw press, a meat and bone separator, a press dehydrator, a centrifuge, and combinations thereof.

The pressed liquid may be heated under an atmospheric, superatmospheric, or subatmospheric pressure at 50° C. or higher, preferably at 70-150° C., particularly preferably at 85-110° C. Upon this heating, a solids content (thermal coagulum) is separated from water-soluble components and filtered, centrifuged or otherwise treated to give a thermal coagulum. The thermal coagulum may be dried as appropriate before use. The drying operation can be performed by either one or combinations of hot air drying, drying with vapor, drying through high-frequency/microwave heating, vacuum/reduced pressure drying, drying through freezing/thawing, and drying with a desiccant. If the drying temperature is unduly high, the lipid is oxidized to give off a malodor; hence, drying is performed at 90° C. or below, preferably at 75° C. or below, more preferably at 55° C. or below. Drying is a preferred process because it allows removal of volatile impurities. Said thermal coagulum or dried product thereof can preferably be applied in the present invention since it contains astaxanthin.

In addition, a step for lowering the residual content of impurities may be carried out. For example, said thermal coagulum or dried product thereof may be washed with water to lower the concentration of water-soluble components. Washing with water can be performed using freshwater or seawater in a volume at least four times, preferably at least ten times, the weight of the dry content in said thermal coagulum or dried product thereof. Preferably, washing is done at least twice, more preferably at least three times. To perform washing, water may be injected into the thermal coagulum or dried product thereof in a vessel and after standing for at least 5 minutes, the water content is separated. Depending on the form of the thermal coagulum or dried product thereof, thorough agitation is also effective. Alternatively, the thermal coagulum or dried product thereof in a vessel may be washed under flushing water.

The krill oil in the present invention contains a phospholipid. As regards the proportion of the phospholipid in the krill oil, the lower limit is at least 10% (w/w), preferably at least 20% (w/w), and more preferably at least 30% (w/w), whereas the upper limit is not greater than 95% (w/w), preferably not greater than 80% (w/w), and more preferably not greater than 60% (w/w), for example, 10-95% (w/w), preferably 20-80% (w/w), and more preferably 30-60% (w/w).

The krill oil in the present invention contains an n-3 polyunsaturated fatty acid. As regards the proportion of the n-3 polyunsaturated fatty acid in the total fatty acid, the lower limit is at least 5% (w/w), preferably at least 10% (w/w), and more preferably at least 15% (w/w), whereas the upper limit is not greater than 60% (w/w), preferably not greater than 50% (w/w), and more preferably not greater than 30% (w/w), for example, 5-60% (w/w), more preferably 10-50% (w/w), and more preferably 15-30% (w/w).

In one embodiment, the krill oil in the present invention contains EPA as the n-3 polyunsaturated fatty acid. As regards the proportion of EPA in the total fatty acid, the lower limit is at least 2% (w/w), preferably at least 5% (w/w), and more preferably at least 10% (w/w), whereas the upper limit is not greater than 40% (w/w), preferably not greater than 30% (w/w), and more preferably not greater than 20% (w/w), for example, 2-40% (w/w), preferably 5-30% (w/w), and more preferably 10-20% (w/w).

In another embodiment, the krill oil in the present invention contains DHA as the n-3 polyunsaturated fatty acid. As regards the proportion of DHA in the total fatty acid, the lower limit is at least 1% (w/w), preferably at least 2.5% (w/w), and more preferably at least 4% (w/w), whereas the upper limit is not greater than 20% (w/w), preferably not greater than 15% (w/w), and more preferably not greater than 10% (w/w), for example, 1-20% (w/w), preferably 2.5-15% (w/w), and more preferably 4-10% (w/w).

In a further embodiment, the krill oil in the present invention contains astaxanthin. Astaxanthin may be present either in a free state or in a lipid state involving an ester linkage. As for the content of astaxanthin in the krill oil, the lower limit is at least 20 ppm, preferably at least 50 ppm, and more preferably at least 100 ppm, whereas the upper limit is not greater than 1000 ppm, preferably not greater than 600 ppm, and more preferably not greater than 400 ppm, for example, 20-1000 ppm, preferably 50-600 ppm, and more preferably 100-400 ppm.

According to another aspect of the present invention, there are provided a brain function improving agent and a cognitive judgment ability improving agent, both containing a phospholipid as an active ingredient. The daily amount of the phospholipid to be ingested is 0.008-160 mg/kg, preferably 0.08-80 mg/kg, and more preferably 1.2-24 mg/kg, per kilogram of an animal's body weight. In particular, in the case of a human adult, the amount of the phospholipid to be ingested is 0.4-8000 mg/50 kg body weight, preferably 4-4000 mg/50 kg body weight, and more preferably 60-1200 mg/50 kg body weight, per day.

Phospholipids are known as a major component of cell membranes and have a hydrophilic phosphate moiety and a hydrophobic fatty acid moiety. Phospholipids are divided into diacylglycerophospholipids, which have the fatty acid moiety at 1- and 2-positioins on the glycerol backbone, and lysoacylglycerophospholipids. Lysoacylglycerophospholipids are divided into 1-acylglycerolysophospholipids having the fatty acid part only at 1-position on the glycerol backbone and 2-acylglycerolysophospholipids having the fatty acid part only at 2-position on the glycerol backbone. Exemplary diacylglylcerophospholipids include phosphatidylcholine (PC), phosphatidylethanolamine (PE), phosphatidylserine (PS), phosphatidylinositol (PI), phosphatidylglycerol (PG), cardiolipin (CL), phosphatidic acid (PA), and mixtures of two or more kinds thereof, preferably PC, PE, PS, PI, PA, and mixtures of two or more kinds thereof, particularly preferably PC, PS, and a mixture thereof. Exemplary lysoacylglycerophospholipids include 1- or 2-lysoPC, 1- or 2-lysoPE, 1- or 2-lysoPS, 1- or 2-lysoPI, 1- or 2-lysoPG, 1- or 2-lysoCL, 1- or 2-lysoPA, and mixtures of two or more kinds thereof, preferably 1- or 2-lysoPC, 1- or 2-lysoPE, 1- or 2-lysoPS, 1- or 2-lysoPI, 1- or 2-lysoPA, and mixtures of two or more kinds thereof, particularly preferably 1- or 2-lysoPC, 1- or 2-lysoPS, and a mixture thereof. The phospholipids as referred to herein embrace any one of these phospholipids, with diacylglycerophospholipids being particularly preferred.

The phospholipids to be used in the present invention have polyunsaturated fatty acids as a constituent fatty acid. The polyunsaturated fatty acids as referred to herein are fatty acids having at least three double bonds and containing at least 18, preferably at least 20, carbon atoms. Preferred polyunsaturated fatty acids are n-3 polyunsaturated fatty acids. The term “n-3 polyunsaturated fatty acids” as referred to herein means a fatty acid in which the third and fourth carbon atoms as counted from the terminal carbon on the opposite side of the carboxyl group of the fatty acid molecule form a double bond. Examples of such fatty acids include eicosapentaenoic acid (20:5, EPA), docosapentaenoic acid (22:5, DPA), docosahexaenoic acid (22:6, DHA), etc., with EPA and DHA being preferred. As regards the proportion of the n-3 polyunsaturated fatty acid in the constituent fatty acids of the phospholipid to be used in the present invention, the lower limit is at least 5% (w/w), preferably at least 10% (w/w), and more preferably at least 15% (w/w), whereas the upper limit is not greater than 90% (w/w), preferably not greater than 80% (w/w), and more preferably not greater than 70% (w/w), for example, 5-90% (w/w), preferably 10-80% (w/w), and more preferably 15-70% (w/w).

The phospholipids to be used in the present invention contain EPA as a constituent fatty acid. As regards the proportion of EPA in the constituent fatty acids of the phospholipid, the lower limit is at least 4% (w/w), preferably at least 10% (w/w), and more preferably at least 20% (w/w), whereas the upper limit is not greater than 60% (w/w), preferably not greater than 50% (w/w), and more preferably not greater than 40% (w/w), for example 4-60% (w/w), preferably 10-50% (w/w), and more preferably 20-40% (w/w).

The phospholipids to be used in the present invention contain DHA as a constituent fatty acid. As regards the proportion of DHA in the constituent fatty acids of the phospholipid, the lower limit is at least 3% (w/w), preferably at least 7.5% (w/w), and more preferably at least 12% (w/w), whereas the upper limit is not greater than 40% (w/w), preferably not greater than 30% (w/w), and more preferably not greater than 20% (w/w), for example 3-40% (w/w), preferably 7.5-30% (w/w), and more preferably 12-20% (w/w). The n-3 polyunsaturated fatty acids have high fluidity and, hence, the more they are contained in the phospholipid, the more effective they are in imparting good properties at low temperature. However, the amount of n-3 polyunsaturated fatty acids that are contained in yet to be purified natural materials is only about 60% at maximum, and to increase their concentration, an extra cost for enrichment will incur.

The phospholipids in the present invention may be prepared from any kinds of materials, including fish and shellfish, animals, egg yolk, plants, and fungi. Specific examples include krill oil, fish oil, fish extract, squid extract, bonito ovary extract, extracts of animals provided with feeds incorporating n-3 polyunsaturated fatty acids or extracts of the egg yolk of such animals, linseed oil, extracts of transgenic plants, etc. and extracts of Labyrinthulea, etc. Among these, krill oil, squid extract and bonito ovary extract, etc. are preferred since they are particularly abundant in phospholipids. Alternatively, these materials themselves may be used as phospholipids in the present invention. By using techniques commonly known in the technical field concerned, such as concentrating, extracting, and/or purifying and blending, phospholipids can be prepared from the above-mentioned materials, with their concentration or purity being adjusted to any desired values.

According to another aspect of the present invention, there is provided a food or beverage that contains the brain function improving agent which is to be explained herein. According to yet another aspect of the present invention, there is provided a food or beverage that contains the cognitive judgment ability improving agent which is also to be explained herein.

According to another aspect of the present invention, there is provided a food or beverage that contains krill oil. Krill oil may be contained in the food or beverage of the present invention in an amount that is effective for improving brain functions and/or cognitive judgment ability. The amount of krill oil to be ingested per meal is 0.02 to 400 mg/kg, preferably 0.2-200 mg/kg, more preferably 3-60 mg/kg, for example, 25-40 mg/kg, per kilogram of an animal's body weight. In particular, in the case of a human adult, the amount of krill oil to be ingested is 1-20000 mg/50 kg body weight, preferably 10-10000 mg/50 kg body weight, particularly preferably 150-3000 mg/50 kg body weight, for example, 1250-2000 mg/50 kg body weight, per meal. In order to ensure that a more marked effect is obtained for improving brain functions and/or cognitive judgment ability in a human adult, the intake is preferably increased but if it is excessive, increased oiliness results to cause undesirable effects including retarded absorption, indigestion, dyspepsia, and avoidance of ingestion. It should also be noted that the food or beverage of the present invention is preferably ingested continuously; for example, it is continuously ingested for at least 7 days, preferably at least 30 days, and more preferably at least 90 days.

According to another aspect of the present invention, there is provided a food or beverage that contains a phospholipid. The phospholipid may be contained in the food or beverage in an amount that is effective for improving brain functions and/or cognitive judgment ability. The amount of phospholipid to be ingested per meal is 0.008 to 160 mg/kg, preferably 0.08-80 mg/kg, and more preferably 1.2-24 mg/kg, per kilogram of an animal's body weight. In particular, in the case of a human adult, the amount of phospholipid to be ingested is 0.4-8000 mg/50 kg body weight, preferably 4-4000 mg/50 kg body weight, and more preferably 60-1200 mg/50 kg body weight, per meal.

The brain function improving agent and the food or beverage of the present invention can be used to improve brain functions. The term “improvement of brain functions” as referred to herein means an improvement, recovery, maintenance, prevention of decline, etc. of brain functions during the execution of tasks. The following effects, for example, can be obtained by ingesting the brain function improving agent or the food or beverage of the present invention:

Brain functions are augmented to thereby enhance the task processing ability;

Brain functions that have declined due to fatigue are recovered to return to the normal state;

Brain functions that have declined due to aging are recovered to thereby promote rejuvenation;

Decline of brain functions due to aging is prevented to thereby retard weakening; or

Brain functions that have declined due to disease are recovered to thereby treat the disease or mitigate its symptoms, thus promoting a return to the social life; the disease as referred to herein means mental disorders such as dementia, dementia-related disease, and depression.

The cognitive judgment ability improving agent and the food or beverage of the present invention can be used to improve cognitive judgment ability. The term “improvement of cognitive judgment ability” as referred to herein means an augmentation, recovery, maintenance, prevention of decline, etc. of cognitive judgment ability. The following effects, for example, can be obtained by ingesting the cognitive judgment ability improving agent or the food or beverage of the present invention:

Cognitive judgment ability is augmented to enhance the task processing speed;

Cognitive judgment ability that has declined due to fatigue is recovered to return to the normal state;

Cognitive judgment ability that has declined due to aging is recovered to thereby promote rejuvenation;

Decline of cognitive judgment ability due to aging is prevented to thereby retard weakening; or

Cognitive judgment ability that has declined due to disease is recovered to thereby treat the disease or mitigate its symptoms, thus promoting a return to the social life; the disease as referred to herein means dementia, dementia-related disease, neurodegenerative disorder, etc.

The brain function improving agent, cognitive judgment ability improving agent, or foods or beverages according to the present invention can be applied to foods and beverages that are routinely ingested by healthy persons or ingested by diseased persons, as exemplified by: processed foods such as instant foods and frozen foods that are made from meat, cereal grains, vegetables, fishes, fruits, milk, or the like; beverages such as alcoholic beverages, tea beverages and soft drinks; or health foods, foods for specified health uses, functional foods, foods with nutrient function claims, foods for special dietary uses, hospital diets, and the like that are to be ingested for specified purposes.

The brain function improving agent, cognitive judgment ability improving agent, or foods or beverages according to the present invention can be applied to humans of any age groups. For example, they can be applied to babies (zero to less than one year old), infants (one to less than six years old), children (six to less than 20 years old), adults (20 to less than 65 years old), and the elderly (65 years and older); more preferably, they can be applied to babies, infants, and the elderly.

The task as referred to herein in connection with the brain function improving agent of the present invention may be exemplified by intellectual tasks that require sustained logical thinking. Examples of such tasks include memorization, calculation, reading a book, writing a composition, dissertation, argumentation, creation, etc., that requires logical thinking. The brain function improving agent and the food or beverage of the present invention are capable of improving functions of the brain as it executes those tasks. Specific modes of use include, for example: brain function improving agents and foods or beverages that are ingested to increase the percentage of examination questions answered correctly; brain function improving agents and foods or beverages for recovering brain functions that have declined due to fatigue, aging, etc.; brain function improving agents and foods or beverages that are ingested by diseased patients in order to suppress a decline in brain functions due to the disease or recover the brain function that has declined on account of the disease; brain function improving agents and foods or beverages to be ingested by persons engaged in intellectual tasks; and foods or beverages to be used for school lunch.

The cognitive judgment ability improving agent and the food or beverage of the present invention can be applied with particular preference in operations that require quick judgment on a new task. Specific modes of use include, for example: cognitive judgment ability improving agents or foods or beverages that are ingested by athletes in order to make cognitive judgment faster than competitors in sports such as baseball, soccer, tennis, table tennis, basketball, ski, rugby, American football, karate, and boxing; cognitive judgment ability improving agents or foods or beverages that are ingested by drivers or assistant drivers in order to detect a change in traffic conditions quickly while driving a vehicle that requires operation control, such as a bicycle, an automobile, an airplane, a train, or a motorcycle, so that they can avoid a traffic accident and safely reach the destination; cognitive judgment ability improving agents or foods or beverages that are ingested by game players in order to perform well at video games such as action game and shooting game; cognitive judgment ability improving agents or foods or beverages that are ingested by people whose cognitive judgment has slowed down due to fatigue, aging, etc. in order to get around a danger quickly enough to avoid an injury that might be caused by falling, collision, etc. so that they can live a sound life; and cognitive judgment ability improving agents or foods or beverages that are ingested by diseased patients in order to suppress a decline in cognitive judgment ability due to the disease or recover the cognitive judgment ability that has declined on account of the disease.

In the above-mentioned specific modes of use, the brain function improving agents, cognitive judgment ability improving agents, or foods or beverages according to the present invention are effective whether they are ingested prior to or during a given act. The brain function improving agents, cognitive judgment ability improving agents, and foods or beverages according to the present invention can be rendered into appropriate forms for a specific mode of use. Examples include sheets, capsules, tablets, chewable tablets, sticks, blocks, liquids, suspensions, syrups, pastes, granules (including dry syrup), capsules (soft and hard capsules), dusts, etc.

The brain function improving agent or foods or beverages of the present invention may be sold with an indication of “improving brain functions” being put on the packaging container, the product's instruction, or the product brochure, and this embodiment is included within the scope of the present invention. In a specific mode, the brain function improving agent or foods or beverages of the present invention may be sold with such an indication as “improving thinking power, reading comprehension, calculating skill, creative power, or ability to memorize” or “improving brain functions that have declined due to fatigue, aging, or disease” being put on the packaging container, the product's instruction, or the product brochure.

In addition, the brain function improving agent and foods or beverages of the present invention may be advertised with an indication of “improving brain functions” being put on TV commercials, advertisements, magazines, etc. and this embodiment is also included within the scope of the present invention. In a specific mode, the brain function improving agent and foods or beverages of the present invention may be advertised with such an indication as “improving thinking power, reading comprehension, calculating skill, creative power, or ability to memorize” or “improving brain functions that have declined due to fatigue, aging, or disease” being put on TV commercials, advertisements, magazines, etc.

The cognitive judgment ability improving agent or foods or beverages of the present invention may be sold with an indication of “improving cognitive judgment ability” being put on the packaging container, the product's instruction, or the product brochure, and this embodiment is included within the scope of the present invention. In a specific mode, the cognitive judgment ability improving agent or foods or beverages of the present invention may be sold with such an indication as “improving cognitive judgment ability in doing sports, riding a bicycle, driving a motorcycle, driving an automobile, flying an airplane, driving a train, or playing a video game” or “improving cognitive judgment ability that has declined due to fatigue, aging, or disease” being put on the packaging container, the product's instruction, or the product brochure.

In addition, the cognitive judgment ability improving agent and foods or beverages of the present invention may be advertised with an indication of “improving cognitive judgment ability” being put on TV commercials, advertisements, magazines, etc. and this embodiment is also included within the scope of the present invention. In a specific mode, the cognitive judgment ability improving agent or foods or beverages of the present invention may be advertised with such an indication as “improving cognitive judgment ability in doing sports, driving a vehicle that requires operation control as exemplified by a bicycle, a motorcycle, an automobile, an airplane, a train, or playing a video game” or “improving cognitive judgment ability that has declined due to fatigue, aging, or disease” being put on TV commercials, advertisements, magazines, etc. and this is also included within the scope of the present invention.

The brain function improving agents, cognitive judgment ability improving agents, and foods or beverages according to the present invention may also contain krill oil containing krill-derived ingredients such as ground krill, milled krill, split krill, etc. They may further contain other ingredients in krill oil, such as astaxanthin, sterols, etc. Astaxanthin is a carotenoid compound commonly found in crustaceans such as crab and shrimp. Astaxanthin may occur either in a free state or in a lipid state involving an ester linkage. Alternatively, astaxanthin may separately be added to the above-described brain function improving agents, cognitive judgment ability improving agents, and foods or beverages; in this case, the lower limit of the amount of astaxanthin to be added is, in a free state, at least 1 ppm, preferably at least 5 ppm, and more preferably at least 10 ppm, whereas the upper limit is not greater than 10000 ppm, preferably not greater than 5000 ppm, and more preferably not greater than 1000 ppm, for example, 1-10000 ppm, preferably 5-5000 ppm, and more preferably 10-1000 ppm.

Astaxanthin is preferred because it contributes to stabilizing polyunsaturated fatty acids as an endogenous antioxidant. It should however be noted that unduly high concentrations of astaxanthin are prone to cause problems with color and flavor. Sterols contribute to the fluidity of lipids, so they can enhance the absorbability of the above-described brain function improving agent, cognitive judgment ability improving agent, and foods or beverages.

According to another aspect of the present invention, there is provided a method of improving brain functions which comprises administering to a human the brain function improving agent or the food or beverage for improving brain functions.

The effect of the present invention for improving brain functions can be evaluated by various methods. Concerning the relationship between brain functions and oxyhemoglobin within the brain, it has been reported that oxyhemoglobin within the brain increases as a calculation task is executed and that the degree of the increase of oxyhemoglobin depends on the form and complexity of the task (Melany M. et al., J. Neural Transm (2009) 116:267-273). It has also been reported that the oxyhemoglobin is the most sensitive marker and the most reliable parameter for brain activation (Masato Fukuda, MEDIX VOL. 39.)

Therefore, the effect of the present invention for improving brain functions can be evaluated by using, for example, oxyhemoglobin within the brain as an index. Oxyhemoglobin can be measured by near-infrared spectroscopy (NIRS). The principle of NIRS is described below. An irradiation probe is placed on the scalp to emit weak near-infrared light of about 2 mW toward the skull; the light undergoes repeated scattering and absorption within the tissue before it reaches the cerebral cortex located at a depth about 20-30 mm below the scalp, from which it returns to the scalp. The reflected light (the returning random reflection) as measured with a detection probe placed at a position 30 mm distant from the irradiated position is influenced by a change in blood volume within the tissue. The near-infrared light is absorbed by hemoglobin while it passes through the brain tissue; since the oxyhemoglobin and deoxyhemoglobin within the blood have different absorption spectra in the near-infrared region, two near-infrared wavelengths (695 nm and 830 nm) are used to measure the change in oxyhemoglobin levels.

In one mode of measuring the oxyhemoglobin during the execution of a task in the present invention, a memory task (2-back test) is imposed on a test subject and the oxyhemoglobin levels in the frontal and fronto-temporal regions of the test subject brain during the execution of the task are measured. In the memory task, the numbers 1 to 9 are randomly displayed on a monitor and the test subject is instructed to press the button immediately only when “3” is displayed 2 steps after the display of an even number (2-back test.) Oxyhemoglobin levels are measured as follows: in a given environment, probe holders each fitted with irradiation probes or detection probes in specified positions are placed in a bilateral symmetrical manner so as to cover the frontal and fronto-temporal regions of the test subject, and the oxyhemoglobin levels are measured by NIRS.

In another mode of measuring the oxyhemoglobin level during the execution of a task in the present invention, a calculation task (Kraepelin test) is imposed on a test subject and the oxyhemoglobin levels in the temporal regions of the test subject brain during the execution of the task are measured. In the Kraepelin test, an instruction is given to add adjacent numbers in a fast and correct manner and write down the ones place digit. Oxyhemoglobin levels are measured as follows: in a given environment, probe holders each fitted with irradiation probes or detection probes in specified positions are placed in a bilateral symmetrical manner so as to cover the frontal and fronto-temporal regions of the test subject, and the oxyhemoglobin levels are measured by NIRS.

For the brain to be active, the supply of glucose and oxygen is essential. In particular, for the brain to execute a task, it is essential that glucose and oxygen be supplied to a site in the brain that is involved in the execution of the task. Oxygen is supplied to the brain in a form bound to hemoglobin, namely, as oxyhemoglobin. According to the present invention, the oxyhemoglobin level within the brain can be elevated and, hence, brain metabolism can be activated. Therefore, according to another aspect of the present invention, there are provided a brain metabolism activator as well as a food or beverage for activating brain metabolism, both on account of elevated levels of oxyhemoglobin within the brain. According to yet another aspect of the present invention, there are provided an activator of brain metabolism during task execution as well as a food or beverage for activating brain metabolism.

Oxyhemoglobin is transported to the brain via blood flow and supplied to the necessary sites in accordance with the activity situation of the brain. This means that an improvement of overall circulation is insufficient for executing the task. Therefore, according to another aspect of the present invention, there are provided an agent for improving brain circulation during task execution as well as a food or beverage for improving brain circulation.

According to another aspect of the present invention, there are provided a method of improving cognitive judgment ability which comprises administering to a human the cognitive judgment ability improving agent or the food or beverage for improving cognitive judgment ability.

The effect of the present invention for improving cognitive judgment ability can be evaluated by various methods. For example, it can be evaluated by measuring event-related potentials. The term “event-related potentials” refers to an electrophysiological representation of modes of cognition or information processing within the brain. P300 is known as one of the components of event-related potentials. Being a component that appears in about 300 milliseconds after the display of a stimulus irrespective of whether it is visible, tactile, audible, olfactory, or gustatory, P300 reflects the process by which a cognitive context is updated. Thus, P300 is said to be a component that reflects the process by which an information processing flow once formed is changed in accordance with a new input of information.

P300 is evaluated by latency and amplitude. Latency means the speed at which a task is processed, and the shorter the latency, the higher the processing speed. In other words, latency reflects the time it takes to make cognitive judgment of a new task. In contrast, amplitude represents the size of processing resources that can be used to execute the task; namely, amplitude means the processing ability, and the greater the amplitude, the higher the ability to execute the task. Therefore, the term “task” as referred to herein embraces every task that provides the brain with a stimulus that causes P300 to appear in event-related potentials.

It is known that the latency of P300 is extended by aging or neurodegenerative disorders (Y. Hirayasu et al., Clinical Neurophysiology, 111 (2000), 187-194) and that further the latency of P300 is related to intellectual executing power, working memory ability, and the task processing speed in healthy persons and patients with neurodegenerative disorders (Emmerson et al., Exp Aging Res, 1989, 15:151-159; O'Donnell et al., Int J Psychophysiol, 1992, 12:187-195; O'Donnell et al., Int J Psychophysiol, 1992,12:187-195). Thus, the latency of P300 is widely used as an index for evaluating cognitive judgment ability.

Therefore, in the present invention, the effect for improving cognitive judgment ability is evaluated using a change in the latency of P300 as an index. In a mode of measuring event-related potentials during the execution of a task in the present invention, a memory task is imposed on a test subject and electroencephalograms of the test subject during the execution of the task are measured to observe changes in the electroencephalograms. In the memory task, the numbers 1 to 9 are randomly displayed on a monitor and the test subject is instructed to press the button immediately only when “3” is displayed 2 steps after the display of an even number (2-back test.) Event-related potentials are measured as follows: in a given environment, measuring electrodes are attached at the sites on the scalp that are specified by the international 10-20 method and event-related potentials are derived using a linked earlobes reference. For a given interval from a time before display of a stimulus to a time after the display, the waveforms of event-related potentials are measured to determine the peak latencies of P300 and the peak amplitudes.

Based on the foregoing, the present invention can provide a method of improving cognitive judgment ability which comprises administering to a human the cognitive judgment ability improving agent or the food or beverage.

The brain function improving agents, cognitive judgment ability improving agents, or foods or beverages according to the present invention may, depending on the need, be used in combination with ingredients conventionally known to be effective in improving brain functions and/or cognitive judgment ability, as exemplified by ginkgo leaf extract, L-carnitine, melatonin, coenzyme Q10, α-lipoic acid, nucleic acids, Ganoderma lucidum (Leyss. ex. Fr.) Karst, ceramide, grape seed extract, polyphenols, pine bark extract, glucosamine, harp seal oil, Hericium erinaceum, Angelica keiskei, garlic extract, Cordyceps sinensis (Berkeley) Saccardo, turmeric, maca, black currant extract, astaxanthin, GABA, α-glycerophosphocholine, arachidonic acid, theanine, etc. In particular, the combination with arachidonic acid is preferred from the viewpoint of adjusting the balance between n-3 fatty acids and n-6 fatty acids. Moreover, depending on the need, other conventionally known ingredients may be contained, as exemplified by coloring agents, preservatives, scents, flavoring agents, coating agents, antioxidants, vitamins, amino acids, peptides, proteins, minerals (e.g., iron, zinc, magnesium, and iodide), etc.

Examples of antioxidants here mentioned include tocopherol, dried yeast, glutathione, lipoic acid, quercetin, catechin, coenzyme Q10, enzogenol, proanthocyanidins, anthocyanidin, anthocyanin, carotenes, lycopene, flavonoid, resveratrol, isoflavones, zinc, melatonin, ginkgo leaf, Alpinia zerumbet leaf, hibiscus, C vitamins, or extracts thereof

In a specific embodiment, tocopherol is incorporated in such a way that its daily intake is 0.50-650 mg/kg body weight, preferably 5-500 mg/kg body weight, and more preferably 50-300 mg/kg body weight.

In another specific embodiment, catechin is incorporated in such a way that its daily intake is 1-5000 mg/kg body weight, preferably 10-3000 mg/kg body weight, and more preferably 100-1000 mg/kg body weight.

In another specific embodiment, C vitamins are incorporated in such a way that their daily intake is 1-1000 mg/kg body weight, preferably 10-800 mg/kg, and more preferably 100-500 mg/kg body weight.

Examples of vitamins include: A vitamins (e.g. retinal, retinol, retinoic acid, carotene, dehydroretinal, lycopene, and salts thereof); B vitamins (e.g. thiamine, thiamine disulfide, dicethiamine, octothiamine, cycothiamine, bisibuthiamine, bisbenthiamine, prosulthiamine, benfothiamine, fursultiamine, riboflavin, flavin adenine dinucleotide, pyridoxine, pyridoxal, hydroxocobalamin, cyanocobalamin, methylcobalamin, deoxyadenocobalamin, folic acid, tetrahydrofolic acid, dihydrofolic acid, nicotinic acid, nicotinic acid amide, nicotinic alcohol, pantothenic acid, panthenol, biotin, choline, inositol, pangamic acid, and salts thereof); C vitamins (ascorbic acid and derivatives thereof, erythorbic acid and derivatives thereof, as well as pharmaceutically acceptable salts thereof); D vitamins (e.g. ergocalciferol, cholecalciferol, hydroxycholecalciferol, dihydroxycholecalciferol, dihydrotachysterol, and pharmaceutically acceptable salts thereof); E vitamins (e.g. tocopherol and derivatives thereof, ubiquinone derivatives, and pharmaceutically acceptable salts thereof); and other vitamins (e.g. carnitine, ferulic acid, γ-oryzanol, orotic acid, rutin (vitamin P), eriocitrin, hesperidin, and pharmaceutically acceptable salts thereof)

Examples of amino acids include leucine, isoleucine, valine, methionine, threonine, alanine, phenylalanine, tryptophan, lysine, glycine, asparagine, aspartic acid, serine, glutamine, glutamic acid, proline, tyrosine, cysteine, histidine, ornithine, hydroxyproline, hydroxylysine, glycylglycine, aminoethylsulfonic acid (taurine), cystine, and pharmaceutically acceptable salts thereof

The present invention permits preparation in forms as suitable for pharmaceutical compositions, functional foods, health foods, and dietary supplements; examples include various solid formulations such as granules (including dry syrup), capsules (soft and hard capsules), tablets (including chewable tablets and the like), powders (dusts), and pills, as well as liquid formulations such as liquids for internal use (including liquids, suspensions, and syrups.)

Examples of additives that may be used to prepare formulations include excipients, lubricants, binders, disintegrants, fluidization agents, dispersants, wetting agents, antiseptics, viscous agents, pH adjusting agents, coloring agents, corrigents, surfactants, and solubilizers. In the case of preparation in the form of liquids, thickening agents such as pectin, xanthan gum, and guar gum may be incorporated. If desired, coating agents may be used to formulate coated tablets or gelatin pastes. Even other forms of preparation may be produced in accordance with conventional methods.

### Examples

The present invention is described more specifically by means of the examples given below but it should be understood that the scope of the present invention is in no way limited by those examples.

### Example 1

### Production of Krill Oil

Antarctic krill (10 tons) as caught was pressed with a meat and bone separator (product of BAADER; model BAADER 605) to give a pressed liquid (3 tons). This pressed liquid was placed in 800-kg portions into stainless steel tanks and then heated by directly feeding in steam at 140° C. The heating was continued for about 60 minutes and after confirming that a temperature of 85° C. had been reached, the heating operation was stopped. The valve on the bottom of each tank was opened to let the liquid component drop under gravity through a screen with a mesh size of 2 mm. The retained solids (thermal coagulum) were washed by showering with an equal volume of water, placed in 12-kg portions into aluminum trays, and rapidly frozen with a contact freezer. The total weight of the resulting thermal coagulum was 2.25 tons.

The frozen product (1 ton) was charged into water (3000 L) which was heated with stirring until a temperature of 65° C. was reached and then held at that temperature for 10 minutes. The mixture was strained through a 24-mesh nylon screen and the retained solids were charged into 3000 L of water (20° C.). After 15-min stirring, the mixture was strained through a 24-mesh nylon screen and treated with a centrifugal dehydrator (centrifuge model 0-30 manufactured by TANABE WILLTEC INC; 15 seconds) to give solids with a water content of about 73%. To the solids, 0.3% of tocopherol was added and after thorough blending with a mixer, the mixture was dried with hot air at 70-75° C. for 3.2 hours to give a washed and dried product (170 kg). The other frozen product was treated in the same manner.

To the washed and dried products (300 kg), 99% ethanol (1200 L) was added and the mixture was heated to 60° C. at which it was stirred for 2 hours. Thereafter, solid-liquid separation was effected by passing the mixture through a 100-mesh nylon screen by gravity fall, to give extract A and cake A. To cake A, 99% ethanol (800 L) was added and the mixture was heated to 60° C. at which it was stirred for 2 hours; thereafter, solid-liquid separation was effected by passing the mixture through a 100-mesh nylon screen to give extract B and cake B. To cake B, 99% ethanol (700 L) was added and the mixture was heated to 60° C. at which it was stirred for 2 hours; thereafter, solid-liquid separation was effected by passing the mixture through a 100-mesh nylon screen to give extract C and cake C. Extracts A, B and C were combined to make a weight of 2021 kg. It was then concentrated under reduced pressure at a liquid temperature of no more than 60° C. so that ethanol and water were distilled off to give a lipid extract (145.0 kg).

### Test Example 1

### (1) Ingestion of Test Samples and the Items of Test

The test subjects were 45 healthy males in their sixties (all of them were right-handed). Prior to the test, the purpose of the research was explained in writing to the test subjects who then consented to participate in the research.

The following samples were ingested by the test subjects.

Sardine oil: Prepared using sardine oil as a raw material through a process comprising extraction, distillation, discoloration, deodorization, degumming, concentrating, etc.

Medium chain fatty acid oil (placebo): Prepared using coconut palm and palm fruit as raw materials through a process comprising enzyme reaction, deacidification, deodorization, etc.

Krill oil: As produced in Example 1.

The basic ingredients of the each sample are shown in Table 1. To analyze the fatty acid composition, each sample was treated by the methyl esterification method (boron trifluoride/methanol method) described in the Standard Methods for Analysis of Fats, Oils and Related Materials (formulated by Japan Oil Chemists' Society), whereupon the fatty acids were converted to methyl esters and the resulting fatty acid methyl esters were detected with a gas chromatograph equipped with a flame ionization detector; the thus obtained area percentages were taken as weight percentages.

TABLE 1
Basic ingredients of samples

| Krill oil | Basic ingredients | |
|---|---|---|
| | Water | 0.2%(w/w) |
| | Lipids | 99.0%(w/w) |
| | Phospholipid content | 44.9%(w/w) |
| | Tocopherol | 0.7%(w/w) |
| | Composition of principal fatty | |
| | acids (in total fatty acid content) | |
| | EPA | 14.0%(w/w) |
| | DHA | 6.9%(w/w) |
| Sardine oil | Basic ingredients | |
| | Water | 0.0%(w/w) |
| | Lipid (triglyceride) | 99.0%(w/w) |
| | Tocopherol | 1.0%(w/w) |
| | Composition of principal fatty | |
| | acids (in total fatty acid content) | |
| | EPA | 28.8%(w/w) |
| | DHA | 14.7%(w/w) |
| Medium chain | Basic ingredients | |
| fatty acid oil | Water | Not greater |
| (placebo) | | than 1%(w/w) |
| | Lipid (triglyceride) | 99.0%(w/w) |

| Composition of principal fatty | |
|---|---|
| acids (in total fatty acid content) | |
| Caprylic acid | 82.9%(w/w) |
| Capric acid | 15.9%(w/w) |
| EPA | 0.0%(w/w) |
| DHA | 0.0%(w/w) |

The test subjects were allocated to three groups each consisting of 15 men:

Krill oil ingestion group (15 men);

Sardine oil ingestion group (15 men);

Medium chain fatty acid oil ingestion group (15 men).

Each sample was continuously ingested by the test subjects for 12 weeks in a daily dose of 2 g (soft gel capsule). Prior to the ingestion of the sample, at the 6^{th} week of sample ingestion, and at the 12^{th} week of sample ingestion, tasks were imposed on the test subjects and the oxyhemoglobin levels within the brain during the execution of the task were measured.

### (2) Tasks Imposed on the Test Subjects

A memory task (2-back test) and a calculation task (Kraepelin test) were imposed on the test subjects described in (1) above. The following were the conditions for each task.

2-Back Test:

The numbers 1 to 9 were randomly displayed on a monitor installed at a distance of 1.5 m and the test subject was so instructed that only when “3” was displayed 2 steps after the display of an even number should he press the button immediately with the first finger of his right hand. The execution time of the task was set at 450 seconds (7 minutes and 30 seconds.)

Kraepelin Test:

Each test subject was so instructed that adjacent numbers on a Kraepelin test paper should be added in a fast and correct manner and that the ones place digit be written down. The execution time of the task was set at 300 seconds (5 minutes.)

### (3) Measurement of Oxyhemoglobin During the Execution of Task

The oxyhemoglobin in the test subjects during the execution of the calculation task was measured by near-infrared spectroscopy (NIRS) under the following conditions:

Environment: room temperature (22° C.)

### Apparatus: Optical Topography System ETG-4000 (Hitachi Medical Corporation)

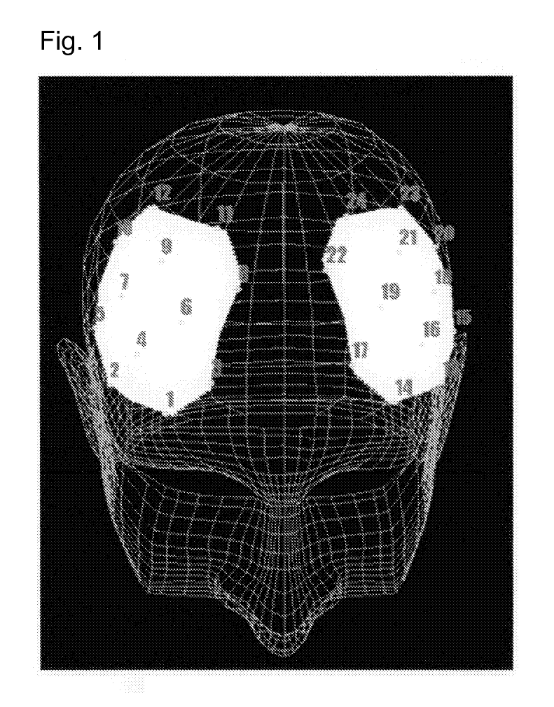

Sites: See FIG. 1. Measurement was conducted in both fronto-temporal regions through 24 channels in total (12 channels on each side). Probe holders (3×3; Hitachi Medical Corporation) were each fitted with irradiation probes or detection probes in specified positions on two surfaces and placed in a bilateral symmetrical manner so as to cover the frontal and fronto-temporal regions of each test subject.

### Durations:

450 seconds in the 2-back test
300 seconds in the Kraepelin test
   Analysis: The changes in oxyhemoglobin levels were determined with the average value for 10 seconds before the start of task execution being taken as a baseline.

It should be noted that the values of total hemoglobin level as measured concurrently were not necessarily in agreement with the trend of changes in oxyhemoglobin levels.

### (4) Results

### (4-1) Changes in Oxyhemoglobin Level During Execution of the 2-Back Test

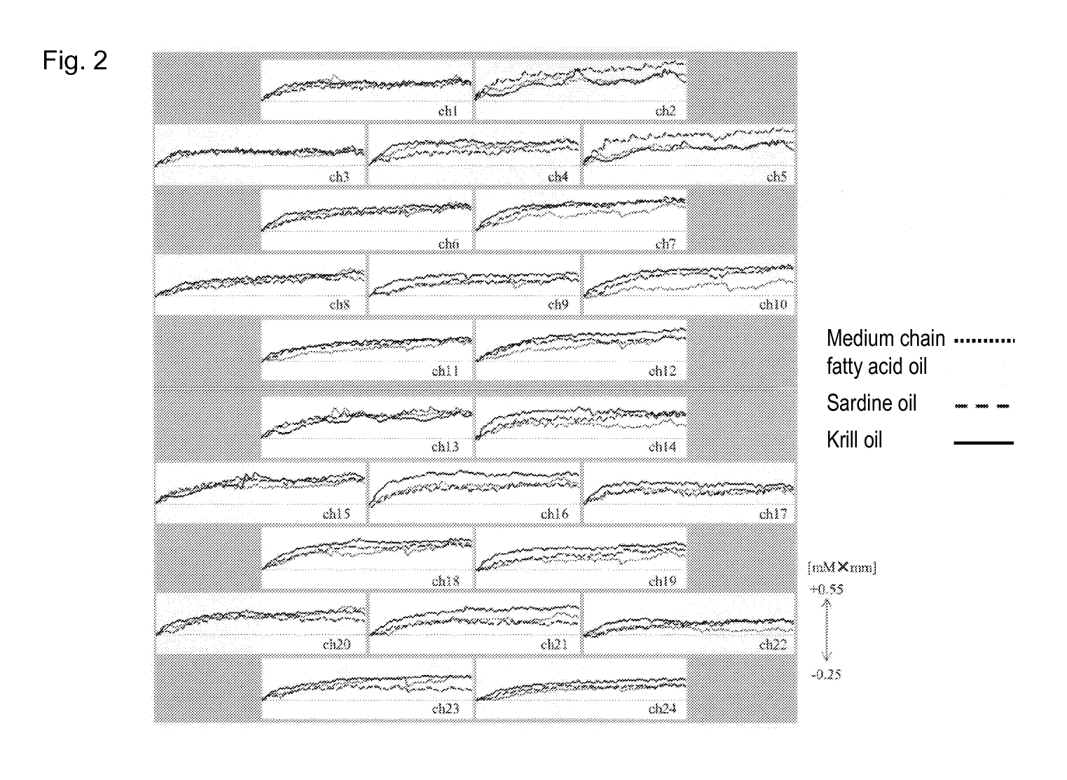

The 2-back test was imposed as a task on each test subject and the changes in oxyhemoglobin level within the brain during execution of the task were investigated. As a typical example of the results, the changes in oxyhemoglobin levels in the test subjects at the 12^{th} week of sample ingestion are shown (FIG. 2.) Considering the features of the task, brain functions were considered to be activated when the oxyhemoglobin level increased from the baseline by at least 0.25 mM×mm. As a general tendency, the oxyhemoglobin level was shown to be higher in the krill oil ingestion group than in the medium chain fatty acid oil ingestion group (placebo) and the sardine oil ingestion group (control). This tendency was especially distinct in ch9, ch10, ch12, ch14, ch16-ch19, and ch21.

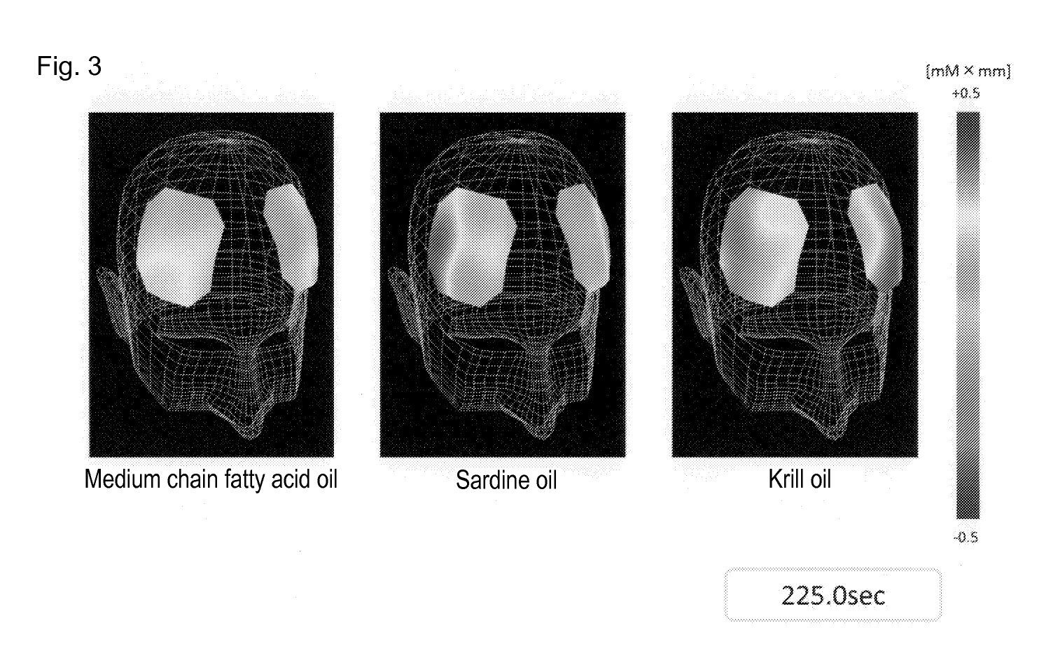

The foregoing results were displayed graphically to visualize the oxyhemoglobin levels in the regions under measurement. As a typical example of the results, the oxyhemoglobin levels in the test subjects at the 12^{th} week after sample ingestion as measured 225 seconds after the start of the 2-back test are shown (FIG. 3.) In the sardine oil ingestion group and the krill oil ingestion group, regions were observed that had higher oxyhemoglobin levels than in the placebo medium chain fatty acid oil ingestion group. In the krill oil ingestion group, the oxyhemoglobin level was higher in all of the regions under measurement. On the other hand, the sardine oil ingestion group had only narrow regions of high oxyhemoglobin level and the level of oxyhemoglobin in other regions was comparable to or lower than the level in the medium chain fatty acid oil ingestion group.

In addition, the measured values of oxyhemoglobin in selected regions (ch10, ch14 and ch19) were extracted and shown below (Table 2.) It is shown that the krill oil ingestion group at the 12^{th} week of sample ingestion has appreciably higher oxyhemoglobin levels than the medium chain fatty acid oil ingestion group. Even in comparison with the sardine oil ingestion group, the krill oil ingestion group had higher oxyhemoglobin levels, with significant differences recognized in ch14 and ch19.

TABLE 2
Changes in oxyhemoglobin level

| Ingestion | | Region under measurement (channel) | | |
|---|---|---|---|---|
| period | Group | ch10 | ch14 | ch19 |
| Week 0 | Medium | 0.213 ± 0.020 | 0.172 ± 0.040 | 0.170 ± 0.041 |
| | chain fatty | | | |
| | acid oil | | | |
| | Sardine oil | 0.173 ± 0.032 | 0.157 ± 0.045 | 0.168 ± 0.051 |
| | Krill oil | 0.206 ± 0.050 | 0.230 ± 0.066 | 0.237 ± 0.051 |
| Week 6 | Medium | 0.258 ± 0.059 | 0.223 ± 0.040 | 0.170 ± 0.044 |
| | chain fatty | | | |
| | acid oil | | | |
| | Sardine oil | 0.194 ± 0.048 | 0.174 ± 0.053 | 0.124 ± 0.051 |
| | Krill oil | 0.217 ± 0.067 | 0.273 ± 0.058 | 0.212 ± 0.046 |
| Week 12 | Medium | 0.102 ± 0.039a | 0.164 ± 0.061a | 0.119 ± 0.038a |
| | chain fatty | | | |
| | acid oil | | | |
| | Sardine oil | 0.250 ± 0.032b | 0.242 ± 0.039a | 0.174 ± 0.032a |
| | Krill oil | 0.306 ± 0.053b | 0.323 ± 0.044b | 0.263 ± 0.050b |

Mean ± S.E. Unlike symbols (a, b) represent a significant difference.

### (4-2) Changes in Oxyhemoglobin Level During Execution of the Kraepelin Test

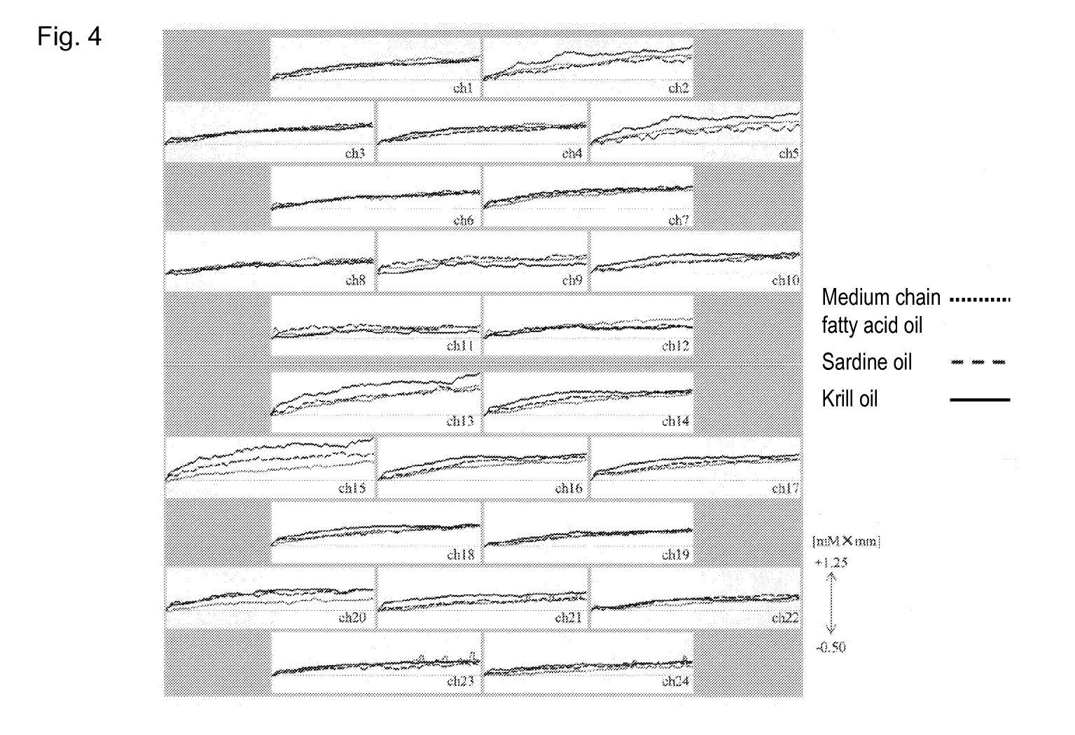

The Kraepelin test was imposed as a task on each test subject and the changes in oxyhemoglobin level within the brain during execution of the task were investigated. As a typical example of the results, the changes in oxyhemoglobin levels in the test subjects at the 12^{th} week of sample ingestion are shown (FIG. 4.) Considering the features of the task, brain functions were considered to be activated when the oxyhemoglobin level increased from the baseline by at least 0.45 mM×mm. As a general tendency, the oxyhemoglobin level was shown to be higher in the krill oil ingestion group than in the medium chain fatty acid oil ingestion group (placebo) and the sardine oil ingestion group (control). This tendency was especially distinct in ch2, ch5, ch10, ch13, ch14-ch18, and ch21.

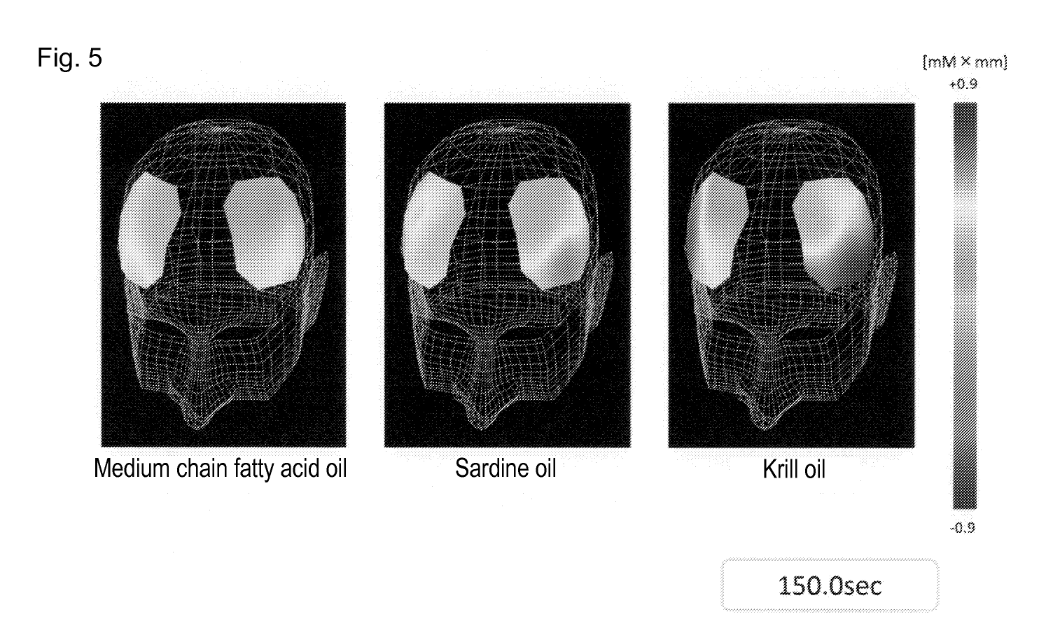

The foregoing results were displayed graphically to visualize the oxyhemoglobin levels in the regions under measurement. As a typical example of the results, the oxyhemoglobin levels in the test subjects at the 12^{th} week of sample ingestion as measured 150.0 seconds after the start of the Kraepelin test are shown in (FIG. 5.) In the sardine oil ingestion group and the krill oil ingestion group, regions were observed that had higher oxyhemoglobin levels than in the placebo medium chain fatty acid oil ingestion group. The krill oil ingestion group was shown to have considerably high oxyhemoglobin levels in a broad range of the regions under measurement. On the other hand, the sardine oil ingestion group did not have as high oxyhemoglobin levels as the krill oil ingestion group and it had only narrow regions of high oxyhemoglobin level.

In addition, the measured values of oxyhemoglobin in a selected region (ch15) were extracted and are shown below (Table 3.) It is shown that the krill oil ingestion group at the 6^{th} and 12^{th} weeks after sample ingestion has higher oxyhemoglobin levels than the medium chain fatty acid oil ingestion group. It is also shown that the krill oil ingestion group at the 6^{th} and 12^{th} weeks after sample ingestion has higher oxyhemoglobin levels than the sardine oil ingestion group.

TABLE 3
Changes in oxyhemoglobin level

| | | Region under |
|---|---|---|
| | | measurement |
| Ingestion | | (channel) |
| period | Group | ch15 |
| Week 0 | Medium chain | 0.315 ± 0.086 |
| | fatty acid oil | |
| | Sardine oil | 0.370 ± 0.108 |
| | Krill oil | 0.521 ± 0.088 |
| Week 6 | Medium chain | 0.464 ± 0.0869 |
| | fatty acid oil | |
| | Sardine oil | 0.356 ± 0.071 |
| | Krill oil | 0.610 ± 0.137 |
| Week 12 | Medium chain | 0.293 ± 0.110a |
| | fatty acid oil | |
| | Sardine oil | 0.556 ± 0.127b |
| | Krill oil | 0.845 ± 0.112b |

Mean ± S.E. Unlike symbols (a, b) represent a significant difference.

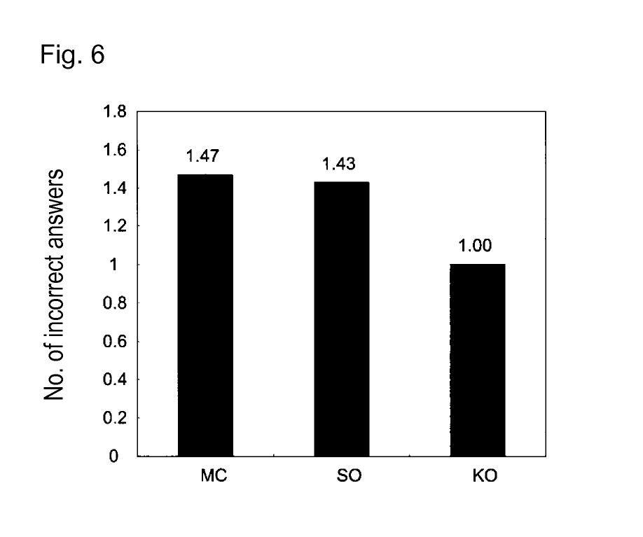

In addition, the number of questions incorrectly answered in the Kraepelin test conducted at week 12 after the start of ingestion of each sample was counted (FIG. 6.) The results showed that the number of incorrect answers was smaller in the krill oil ingestion group than in the medium chain fatty acid oil ingestion group and the sardine oil ingestion group.

From the foregoing, it is clear that the ingestion of krill oil helps to elevate the oxyhemoglobin level within the brain during execution of tasks. It is speculated that the elevated level of oxyhemoglobin within the brain activated brain metabolism to improve brain functions. As a matter of fact, the number of questions answered incorrectly in the task decreased, indicating that the ability to execute the task increased noticeably.

### Test Example 2

### (1) Ingestion of Test Samples and the Item of Test

The test subjects were 45 healthy males in their sixties (right-handed). Prior to the test, the purpose of the research was explained in writing to the test subjects who then consented to participate in the research. The test subjects were allowed to ingest the same samples as used in Test 1.

The test subjects were divided into three groups each consisting of 15 men:

Krill oil ingestion group (15 men);

Sardine oil ingestion group (15 men);

Medium chain fatty acid oil ingestion group (15 men).

Each sample was continuously ingested for 12 weeks in a daily dose of 2 g (soft capsule). Prior to the ingestion of the sample, at the 6^{th} week after sample ingestion, and at the 12^{th} week after sample ingestion, a task was imposed on the test subjects and the event-related potentials during the execution of the task were measured.

### (2) Task

A memory task (2-back test) was imposed on the test subjects described in (1) above. The following were the conditions for the task.

The numbers 1 to 9 were randomly displayed on a monitor installed at a distance of 1.5 m and the test subject was so instructed that only when “3” was displayed 2 steps after the display of an even number should he press the button immediately with the first finger of his right hand. The execution time of the task was set at 7 minutes and 30 seconds.

### (3) Measurement of the Event-Related Potentials During Task Execution

The electroencephalograms during memory task execution were measured in each test subject and the changes in the electroencephalograms were observed. The measurement was conducted under the following conditions:

Environment: room temperature (22° C.)
Sites: In accordance with the international 10-20 method, Ag/AgCl electrodes were attached at two sites (Cz and Pz) on the scalp and event-related potentials were derived using a linked earlobes reference.
Duration: 450 seconds (7 minutes and 30 seconds)
Analysis: For an interval from 100 ms before the presentation of a stimulus to 900 ms after the presentation, 20-30 trials were averaged to acquire the waveforms of event-related potentials. The P300 component was noted and its peak latency and peak amplitude were determined

### (4) Results

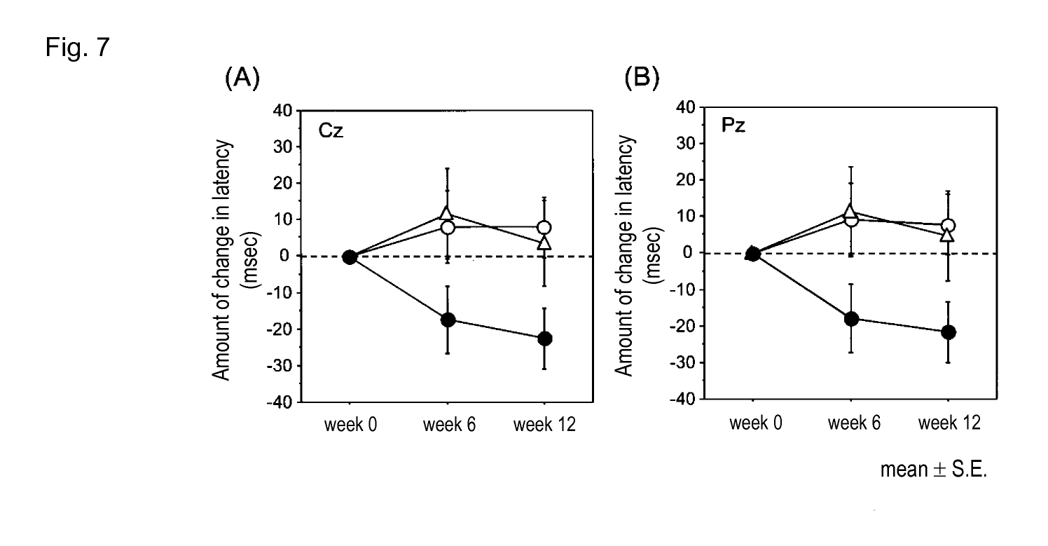

Based on the waveforms of event-related potentials as acquired from the test subjects in the medium chain fatty acid oil ingestion group, the sardine oil ingestion group, and the krill oil ingestion group, the peak latencies of P300 from the test subjects before the start of ingestion, at the 6^{th} week of ingestion, and at the 12^{th} week of ingestion were measured in each ingestion group and the amounts of mean changes in the P300 peak latencies were calculated with reference to the values recorded before the start of ingestion (FIG. 7.)

At Cz, the test subjects in the krill oil ingestion group experienced considerable shortening of the P300 peak latency (FIG. 1A). As compared with the data before ingestion, the P300 peak latency for the test subjects in the krill oil ingestion group was shortened by 17.4 milliseconds on average at the 6^{th} week of sample ingestion and 22.6 milliseconds on average at the 12^{th} week of sample ingestion. This effect for shortening the P300 peak latency was shown to have a significant difference in connection with the medium chain fatty acid oil ingestion group. On the other hand, no changes in the P300 peak latency could be verified for the test subjects in the sardine oil ingestion group and the medium chain fatty acid oil ingestion group.

At Pz, too, the test subjects in the krill oil ingestion group experienced considerable shortening of the P300 peak latency (FIG. 1B). As compared with the data before ingestion, the P300 peak latency for the test subjects in the krill oil ingestion group was shortened by 18.0 milliseconds on average at the 6^{th} week of sample ingestion and 21.7 milliseconds on average at the 12^{th} week of sample ingestion. This effect for shortening the P300 peak latency was shown to have a significant difference in connection with the medium chain fatty acid oil ingestion group.

While the averaged peak latencies of P300 at the 12^{th} week of the ingestion of krill oil were as noted above, an observation of the individual test subjects verified that the degree by which the P300 peak latency was shortened was at least 3 milliseconds and 70 milliseconds at maximum.

It has been reported that in humans over the age of 20, the P300 latency increases at a rate of 1 millisecond/year. Therefore, the cognitive judgment ability improving agents and foods or beverages of the present invention are expected to prove highly effective for people over the age of 20, in particular, elderly people.

1-10. (canceled)

11. A method for improving a brain function comprising administering to a healthy subject an effective amount of a phospholipid, wherein the content of an n-3 polyunsaturated fatty acid accounts for at least 5% (w/w) of the constituent fatty acids in the phospholipid.

12. The method according to claim 11, which is for brain metabolism activation and/or cognitive judgment ability improving.

13. The method according to claim 11, which is for improving of brain functions that have declined due to fatigue or aging.

14. The method according to claim 11, comprising administering a krill oil as the phosphorlipid.

15. The method according to claim 14, wherein the daily dose of the krill oil is 0.02-400 mg/kg body weight/day.

16. The method according to claim 14, wherein the daily dose of the krill oil is 10-200 mg/kg body weight/day.

17. The method according to claim 14, wherein the krill oil is purified via a thermal coagulum of krill.

18. The method according to claim 14, wherein astaxanthin content of the krill oil is not more than 1000 ppm.

19. The method according to claim 11, wherein the phospholipid is contained in a food or beverage.
